(19) United States
(12) Patent Application Publication
(10) Pub. No.: US 2024/0368354 A1
(43) Pub. Date: Nov. 7, 2024

(54) STEROID ACID-BASED HYDROGELS

(71) Applicant: DEFENCE THERAPEUTICS INC., Vancouver (CA)

(72) Inventor: Simon Beaudoin, Sherbrooke (CA)

(21) Appl. No.: 18/686,538

(22) PCT Filed: Aug. 26, 2022
(86) PCT No.: PCT/CA2022/051298

§ 371 Date: Feb. 26, 2024

Related U.S. Application Data
(60) Provisional application No. 63/260,648, filed on Aug. 27, 2021.

(51) Int. Cl. C08J 3/075; C07J 9/00; C07K 14/435

(52) U.S. Cl. CPC C08J 3/075; C07J 9/00; C07K 14/43572; C07K 2319/09

(57) ABSTRACT

Steroid acid-based hydrogels and methods of production thereof are described herein. More specifically, the present description relates to steroid acid-peptide conjugates for the formation of a hydrogel. Peptides are covalently conjugated to a one or more of steroid acid moieties and dissolved or resuspended in a solvent to form a hydrogel. The steroid acids may include bile acids and bile acid analogs. The peptides may include a nuclear localisation signal (NLS).

The present description relates to steroid acid-based hydrogels and methods of production thereof. More specifically, the present description relates to steroid acid-peptide conjugates for the formation of a hydrogel.

### BACKGROUND

Hydrogels, or hydrophilic gels, are cross-linked polymer networks that are able to swell and retain a significant amount of water within its structure, but that does not dissolve in water. Hydrogels are commonly used in various industries and have a wide variety of applications such as in cosmetics (e.g., hygienic products), agriculture, pharmaceuticals and therapeutics (e.g., drug delivery systems), biomedical applications, food additives, and mining (e.g., coal dewatering). Novel hydrogels that are easy to produce and cost-effective, as well as low-cost and streamlined methods for producing hydrogels, would be highly desirable.

### SUMMARY

In a first aspect, described herein is a method of forming/producing a hydrogel, the method comprising resuspending/dissolving a peptide covalently conjugated to one or more steroid acid moieties in a solvent until a hydrogel is formed.

In further aspects, described herein is a method of forming/producing a hydrogel, the method comprising providing a peptide to be modified, covalently conjugating the peptide to one or more steroid acid moieties to produce a steroid acid-peptide conjugate, and resuspending/dissolving the steroid acid-peptide conjugate in an solvent until a hydrogel is formed.

A method of forming/producing a hydrogel, the method comprising resuspending/dissolving a peptide in a steroid acid solution, or mixing a steroid acid and peptide in a solvent, until a hydrogel is formed.

In further aspects, described herein is a hydrogel formed/produced by the method described herein.

In further aspects, described herein is a hydrogel comprising the steroid acid-peptide conjugate as defined herein.

In further aspects, described herein is a steroid acid-peptide conjugate, which is the steroid acid-conjugate as defined herein, for use in the production/formation of a hydrogel.

In further aspects, described herein is a use of the steroid acid-conjugate as defined herein, for the production/formation of a hydrogel.

In further aspects, described herein is a steroid acid-peptide conjugate comprising lithocholic acid (LCA), cholic acid (CA), glycochenodeoxycholic acid (GCDCA), glycodeoxycholic acid (GDCA), or glycoursodeoxycholic acid (GUDCA) conjugated to a nuclear localisation signal (NLS) from heterogeneous nuclear ribonucleoprotein (hnRNP) A1 M9 protein, for use in the production/formation of a hydrogel.

In further aspects, described herein is a hydrogel produced/formed by conjugating lithocholic acid (LCA), cholic acid (CA), glycochenodeoxycholic acid (GCDCA), glycodeoxycholic acid (GDCA), or glycoursodeoxycholic acid (GUDCA) to a nuclear localisation signal (NLS) from heterogeneous nuclear ribonucleoprotein (hnRNP) A1 M9 protein.

In further aspects, described herein is a hydrogel comprising a steroid acid-peptide conjugate, wherein the steroid acid-peptide conjugate is or comprises lithocholic acid (LCA), cholic acid (CA), glycochenodeoxycholic acid (GCDCA), glycodeoxycholic acid (GDCA), or glycoursodeoxycholic acid (GUDCA) conjugated to a nuclear localisation signal (NLS) from heterogeneous nuclear ribonucleoprotein (hnRNP) A1 M9 protein.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows the steroid acid-peptide conjugates and peptides alone (control) that were tested for hydrogel formation and their sequences.

FIG. 2 shows the hydrogel formation instantly after dissolution of 25 mg/ml LCA-hnRNPA1 M9 NLS in PBS at room temperature.

FIG. 3 shows the hydrogel formation following 3 hours of incubation of 20 mg/ml CA-, GCDCA-, LCA-, GDCA-, GUDCA-hnRNPA1 M9 NLS in PBS at room temperature.

FIG. 4 shows the hydrogel formation following 1-2 days of incubation of 10 mg/ml GCDCA-, GDCA-, GUDCA-hnRNPA1 M9 NLS in PBS at room temperature.

FIG. 5 shows the absence of hydrogel formation following 1-2 days of incubation of steroid acid-PQBP-1 NLS conjugates at 10-20 mg/ml in PBS at room temperature.

FIG. 6 shows the absence of hydrogel formation following 1-2 days of incubation of steroid acid-GWG-SV40 NLS conjugates at 10-20 mg/ml in PBS at room temperature.

FIG. 7 shows the hydrogel formation following 1-3 hours of incubation of GUDCA-hnRNPA1 NLS conjugate at 20 mg/ml in pure water at room temperature. All steroid acid-hnRNPA1 NLS induced hydrogel formation overnight.

FIG. 8 shows the hydrogel formation following 2 hours of incubation of GCDCA/GDCA/GUDCA-hnRNPA1 NLS conjugates (final concentration 6.67 mg/ml) in complete DMEM containing human cells at room temperature and 37° C.

FIG. 9 shows the hydrogel formation following overnight incubation (37° C. in a thermomixer; 100 rpm) of dimerized CA-NLS1 RPS17 in either methanol:water (40:60), DMSO, or water (Left=pre-incubation; Right=post-incubation).

FIG. 10 shows light microscopy images of the hydrogel formed by dimerized CDCA-hnRNPA1 M9 NLS following overnight incubation (37° C. in a thermomixer; 1000 rpm).

FIG. 11 shows the hydrogel formation following incubation of dimerized in CA-Mellitin (100 mg/ml in DMSO) following overnight incubation (37° C. in a thermomixer; 1000 rpm).

FIG. 12 shows light microscopy images of the hydrogel formed by dimerized CA-Mellitin following overnight incubation (37° C. in a thermomixer; 1000 rpm).

FIG. 13 shows the results of a cytotoxicity assay on breast JIMT1 cancer cells incubated with either dissolved CDCA-hnRNPA1 M9 NLS monomers or dimers (dissolved in DMSO and diluted in cell medium).

FIG. 14 shows the results of a cytotoxicity assay on breast JIMT1 cancer cells incubated with either dissolved CA-Mellitin monomers or dimers (dissolved in DMSO and diluted in cell medium).

FIG. 15 shows the results of a cytotoxicity assay on cells incubated with either CA-NLS1 RPS17 monomers (in DMSO) or dimers dissolved in either H₂O or DMSO.

### SEQUENCE LISTING

This application contains a Sequence Listing in computer readable form created Aug. 27, 2022. The computer readable form is incorporated herein by reference.

TABLE 1
Sequence Listing Description

| SEQ ID NO: | Description |
|---|---|
| 1 | SV40NLS |
| 2 | Nuclear localization signal from SV40 large T-antigen |
| 3 | GWG-SV40NLS |
| 4 | hnRNPA1 M9 NLS |
| 5 | hnRNP D NLS |
| 6 | PQBP-1 NLS |
| 7 | NLS2 RPS17 |
| 8 | NLS3 RPS17 |
| 9 | NLS1 RPS17 |
| 10 | nucleoplasmin NLS |
| 11 | cMyc NLS |
| 12 | TUS NLS |
| 13 | hnRNP M NLS |
| 14 | HuR NLS |
| 15 | NLS2 RG RSP17 |
| 16 | Mellitin |

### DETAILED DESCRIPTION

Described herein are hydrogels and methods relating to producing or forming hydrogels by dissolving or resuspending a steroid acid-peptide conjugate in a solvent. In some aspects, the present invention stems from the demonstration herein that conjugating a peptide (e.g., a peptide comprising nuclear localisation signal [NLS]) to a steroid acid moiety triggers hydrogel formation.

In a first aspect, described herein is a method for producing or forming hydrogels. The method generally comprises selecting/providing a suitable peptide to be modified, and covalently conjugating the peptide to one or more steroid acid moieties to produce a steroid acid-peptide conjugate. In some embodiments, the peptide is conjugated to a number of steroid acid moieties sufficient to improve or promote hydrogel formation.

In some aspects, the method described herein comprises resuspending or dissolving a suitable steroid acid-peptide conjugate in a solvent until a hydrogel is formed. In some embodiments, the concentration of the steroid acid-peptide conjugate dissolved in the solvent or comprised in the hydrogel is at least 0.001, 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL.

In some aspects, the method described herein comprises resuspending or dissolving a suitable peptide in a steroid acid solution until a hydrogel is formed. In some aspects, the peptide and steroid acid spontaneously form a cross-linking polymer network in situ. In some aspects, the peptide and steroid acid are simultaneously or separately added to a solvent.

As used herein, “hydrogel”, or hydrophilic gels, are hydrophilic cross-linked polymer networks that are highly absorbent. The hydrogel defined herein may be of any structure, size, weight, or shape. In some cases, the hydrogel may be one complete hydrogel structure. In some cases, the hydrogel comprises several smaller hydrogel structures. In some cases, the hydrogel is a gelatinous precipitate, or any hydrophilic gel that is opaque or translucent. In some cases, the hydrogel comprises or consists of hydrogel droplets.

In some aspects, the hydrogel described herein is formed or produced spontaneously upon resuspension or dissolution of the peptide and the steroid acid or steroid acid-peptide conjugate in the solvent. In some aspects, the hydrogel described herein is formed or produced instantly upon resuspension or dissolution of the peptide and steroid acid or steroid acid-peptide conjugate in the solvent. In some aspects, the hydrogel is formed or produced at least 1, 5, 10, 15, 20, 25, 30, 35, 40, 45, 50, 55, or 60 seconds after resuspension/dissolution of the peptide and steroid acid or steroid acid-peptide conjugate in the solvent. In some aspects, the hydrogel is formed or produced at least 1, 5, 10, 15, 20, 25, 30, 35, 40, 45, 50, 55, 60, 120, 180, 240, 300, or 360 minutes after resuspension/dissolution of the peptide and steroid acid or steroid acid-peptide conjugate in the solvent. In some aspects, the hydrogel is formed or produced at least 6, 8, 10, 12, 14, 16, 18, 20, 22, or 24 hours after resuspension/dissolution of the peptide and steroid acid or steroid acid-peptide conjugate in the solvent.

As used herein, “peptide” refers to a chain of amino acids of any length, but generally between 7 and 50 amino acids long. In some embodiments, the peptide may comprise one or more synthetic or non-natural amino acids, or modified amino acids. In some embodiments, the peptide is or comprises an NLS or a portion/domain from a polypeptide or an NLS.

As used herein, “solvent” refers to any solution. In some embodiments, the solution or solvent may be an aqueous solution or solvent which comprises water. In some aspects, the solvent is any commonly known buffered solutions. In some cases, solvent is 100% water. The solvent may also be a saline solution, such as but not limited to phosphate buffered saline (PBS) or Tris buffered saline (TBS). The solvent may be a DMSO-water mixture or alcohol-water mixture (e.g., methanol in water) at any ratio. In some aspects, the solvent is not an aqueous solvent (e.g., 100% DMSO). The solvent may be at any pH. In some aspects, the pH of the aqueous solvent is substantially neutral. In some aspects, the pH of the solvent is 6.5, 6.6, 6.7, 6.8, 6.9, 7.0, 7.1, 7.2, 7.3, 7.4, or 7.5.

In some embodiments, the peptides described herein may comprise (or may be engineered to comprise) between 1 to 50, 2 to 50, 5 to 50, or 10 to 50 functional groups (e.g., lysine and/or cysteine residues; or any other group) available for conjugation to the steroid acid moieties described herein. In some embodiments, the peptide antigen may comprise an amino acid sequence that is at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some embodiments, the peptide is conjugated to the steroid acid at solvent-accessible amine (e.g., primary amine) and/or sulfhydryl groups of the peptide. In some embodiments, the peptide is conjugated to the steroid acid via a linker (e.g., bifunctional, trifunctional linker, or multi-functional linker) or at any other chemical or functional group present on or engineered into the peptide. In some embodiments, the peptide is conjugated to the steroid acid-peptide conjugate via an N-terminal or C-terminal cysteine residue. In some embodiments, the peptide comprises a C-terminal amide.

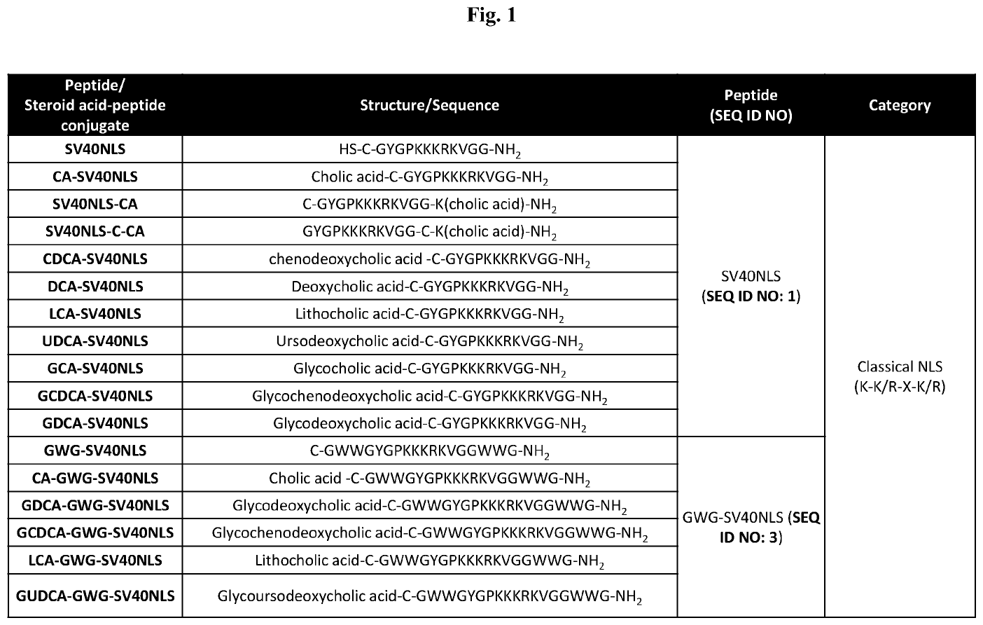

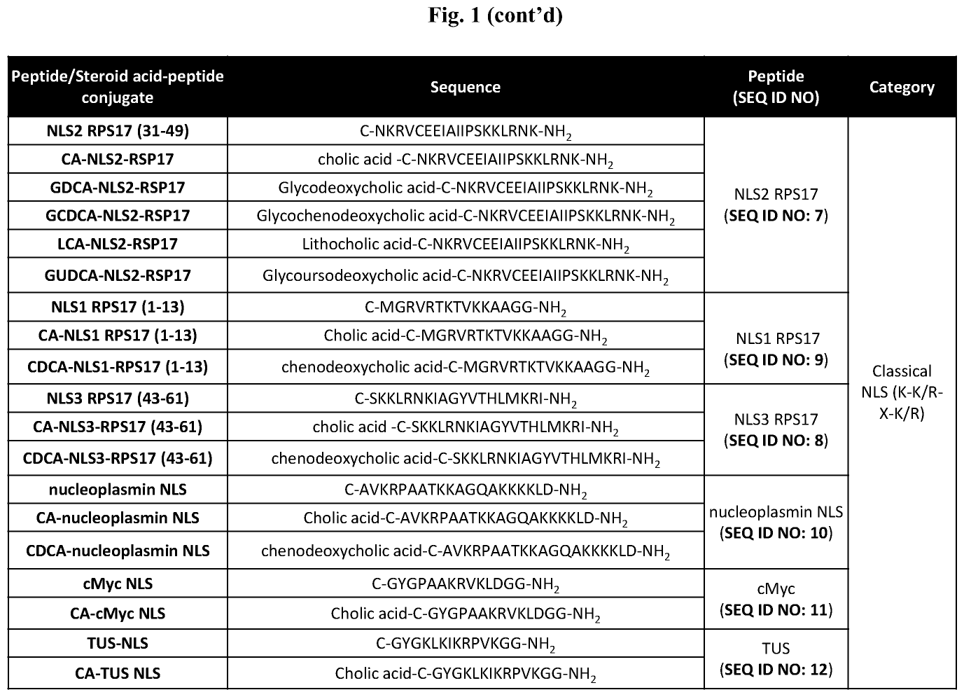

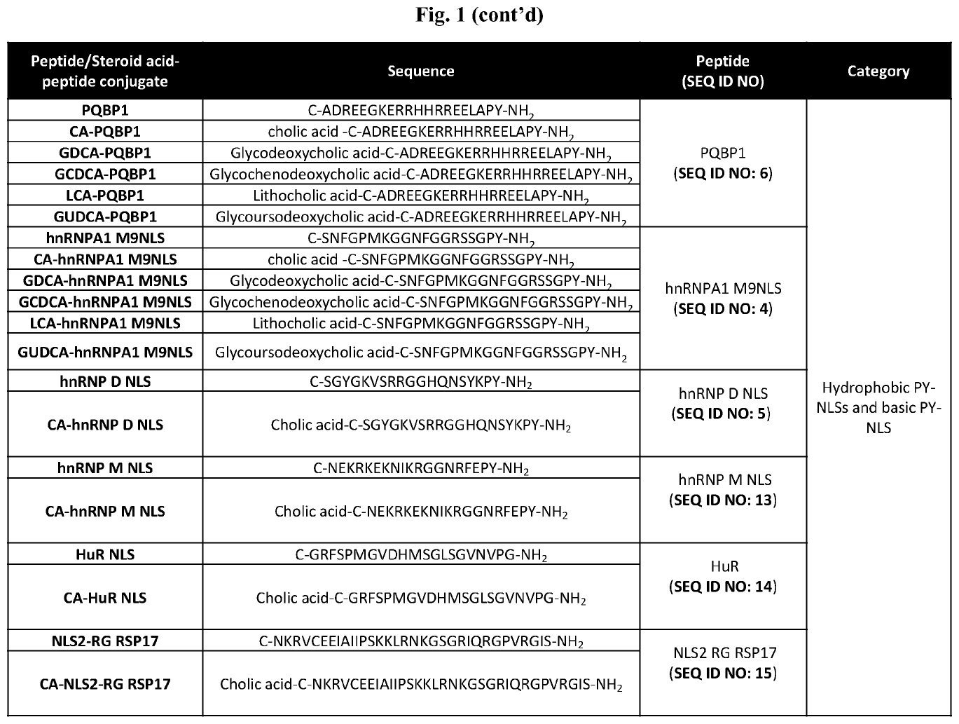

In some embodiments, a steroid acid suitable for conjugation to a peptide described herein comprises or consists of a bile acid (e.g., a primary bile acid or a secondary bile acid). In some embodiments, the steroid acid may be or comprise: cholic acid (CA), chenodeoxycholic acid (CDCA), deoxycholic acid (DCA), lithocholic acid (LCA), glycocholic acid (GCA), taurocholic acid (TCA), glycodeoxycholic acid (CDCA), glycochenodeoxycholic acid (GCDCA), taurodeoxycholic acid (TDCA), glycolithocholic acid (GLCA), taurolithocholic acid (TLCA), taurohyodeoxycholic acid (THDCA), taurochenodeoxycholic acid (TCDCA), ursocholic acid (UCA), tauroursodeoxycholic acid (TUDCA), glycoursodeoxycholic acid (GUDCA), ursodeoxycholic acid (UDCA), or any analog thereof. In some embodiments, the steroid acids described herein are selected from FIG. 1.

In some embodiments, the average number of steroid acid moieties per peptide may be, for example, based on the type of steroid acid and/or type of peptide selected (e.g., amino acid length, structure, number of available functional groups). In some embodiments, the peptide may be reacted with a molar excess of steroid acid or steroid acid-peptide moieties to maximize the number of steroid acid moieties conjugated. In some embodiments, the peptide may be reacted with a limiting amount of steroid acid or steroid acid-peptide moieties to control or limit the number of steroid acid moieties conjugated. In some embodiments, each peptide molecule may be conjugated to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 steroid acid moieties.

In some embodiments, the steroid acids described herein may be comprised in a steroid acid-peptide moiety. In some embodiments, the steroid acid may be pre-conjugated to the peptide, for example at a free N-terminal amino group of the peptide or at some other functional group within the peptide. In some embodiments, the polypeptide antigen may then be conjugated to the steroid acid-peptide moiety via the peptide, such as at an N-terminal cysteine residue of the peptide.

In some embodiments, the peptide may be a non-immunogenic peptide. In some embodiments, the peptide may be a water-soluble peptide, wherein conjugation of the peptide to the steroid acid increases the water solubility of the steroid acid-peptide moiety as compared to the steroid acid moiety alone. In some embodiments, the peptide may be water-insoluble. In some embodiments, the peptide may be a cationic, anionic, or uncharged peptide

In some embodiments, the peptide may comprise one or more domains that impart an additional functionality to the modified polypeptide antigen. As used herein, a “domain” generally refers to apart of a protein having a particular functionality. Some domains conserve their function when separated from the rest of the protein, and thus can be used in a modular fashion. The modular characteristic of many protein domains can provide flexibility in terms of their placement within the peptides described herein. However, some domains may perform better when engineered at certain positions of the peptide (e.g., at the N- or C-terminal region, or therebetween). The position of the domain within its endogenous protein may be an indicator of where the domain should be engineered within the peptide.

In some embodiments, the peptide may comprise a protein transduction domain (PTD) that stimulates endocytosis, endosomal formation, or intracellular delivery in a non-cell-specific manner. In some embodiments, the peptide may comprise a subcellular targeting signal promoting targeting of the modified polypeptide antigen to a specific subcellular compartment. In some embodiments, the peptide may comprise a nuclear localization signal (NLS) that targets the modified polypeptide antigen to the nucleus. In some embodiments, the nuclear localization signals described herein may comprise or be derived from the NLS from SV-40 large T-antigen (e.g., PKKKRKV; SEQ ID NO: 7) or from other classical NLSs. In some embodiments, the nuclear localization signals described herein may comprise or be derived from non-classical NLS (e.g., acidic M9 domain in the hnRNP A1 protein; the sequence KIPIK in yeast transcription repressor Matα2; PY-NLS; ribosomal NLS; or the complex signals of U snRNPs), a hydrophobic PY-NLS, or a basic PY-NLS.

In some embodiments, the nuclear localization signals described herein may comprise the general consensus sequence: (i) K(K/R)X(K/R); (ii) (K/R)(K/R)X₁₀₋₁₂(K/R)_{3/5}, wherein (K/R)_{3/5} represents three lysine or arginine residues out of five consecutive amino acids; (iii) KRX₁₀₋₁₂KRRK; (iv) KRX₁₀₋₁₂K(K/R)(K/R); or (v) KRX₁₀₋₁₂K(K/R)X(K/R), wherein X is any amino acid (Sun et al., 2016).

In some embodiments, the peptide described herein is or comprises a nuclear localisation signal which is an NLS from simian vacuolating virus 40 (SV40) large T-antigen, GWG-SV40NLS, NLS2 from ribosomal protein S17 (RPS17), NLS1 from RPS17, NLS3 from RPS17, NLS2 RG RSP17, nucleoplasmin NLS, acidic M9 domain in the heterogeneous nuclear ribonucleoprotein (hnRNP) A1 protein, hnRNPA1 M9 NLS, hnRNP D NLS, hnRNP M NLS, HuR NLS, cMyc NLS, TUS NLS, polyglutamine-binding protein 1 (PQBP1) NLS, the sequence KIPIK in yeast transcription repressor Matα2, PY-NLS, ribosomal NLS, the complex signals of U snRNPs, or any portion of the NLS thereof.

In some embodiments, the peptide is or comprises a nuclear localisation signal (NLS) comprising an amino acid sequence that at least 70%, 75%, 80%, 85%, 90%, 95%, 99%, or 100% identical to any one of SEQ ID NOs: 1-15 or the sequences as described in FIG. 1.

In some embodiments, the steroid acid-peptide conjugates described herein are selected from FIG. 1.

In some embodiments, the peptide described herein is or comprises Mellitin (SEQ ID NO: 16).

In some embodiments, the steroid acid-peptide conjugate described herein is a monomer or dimer.

In some embodiments, the method for producing/forming the hydrogel described herein further comprises dimerizing the steroid acid-peptide conjugate. Dimerization techniques are commonly known and may include the addition of Diisopropylethylamine (DIPEA) or any other compound/solvent capable of inducing dimerization of peptides.

In some aspects, the peptide, steroid acid or a salt thereof, or steroid acid-peptide conjugate is in solid form, such as a powder or is lyophilized. In some aspects, the final concentration of the steroid acid/peptide mixture or steroid acid-peptide conjugate resuspended/dissolved in the solvent is at least 0.001, 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL.

In some aspects, described herein is a hydrogel formed or produced by the methods described herein.

In some aspects, described herein is a hydrogel comprising the steroid acid-peptide conjugate or steroid acid and peptide mixture defined herein.

In some aspects, described herein is a hydrogel produced/formed by conjugating lithocholic acid (LCA), cholic acid (CA), glycochenodeoxycholic acid (GCDCA), glycodeoxycholic acid (GDCA), or glycoursodeoxycholic acid (GUDCA) to a nuclear localisation signal (NLS) from heterogeneous nuclear ribonucleoprotein (hnRNP) A1 M9 protein to form a conjugate and dissolving/resuspending said conjugate in an solvent.

In some aspects, described herein is a hydrogel comprising a steroid acid-peptide conjugate, wherein the steroid acid-peptide conjugate is or comprises lithocholic acid (LCA), cholic acid (CA), glycochenodeoxycholic acid (GCDCA), glycodeoxycholic acid (GDCA), or glycoursodeoxycholic acid (GUDCA) conjugated to a nuclear localisation signal (NLS) from heterogeneous nuclear ribonucleoprotein (hnRNP) A1 M9 protein.

In some aspects, described herein is a steroid acid-peptide conjugate comprising lithocholic acid (LCA), cholic acid (CA), glycochenodeoxycholic acid (GCDCA), glycodeoxycholic acid (GDCA), or glycoursodeoxycholic acid (GUDCA) conjugated to a nuclear localisation signal (NLS) from heterogeneous nuclear ribonucleoprotein (hnRNP) A1 M9 protein, for use in the production/formation of a hydrogel.

In some aspects, described herein is a steroid acid-peptide conjugate, which is the steroid acid-conjugate as described herein, for use in the production/formation of a hydrogel.

In some aspects, described herein is a use of the steroid acid-conjugate as described herein, for the production/formation of a hydrogel.

### Examples

### Example 1: General Materials and Methods

### Generation of the Steroid Acid-Peptide Conjugates

Steroid acid-peptide conjugates were synthesized similar to the synthesis of cholic acid-NLS (CA-NLS) as previously described in Beaudoin et al., 2016; U.S. Pat. No. 11,291,717; and PCT/CA2022/050714. For example, for CA-SV40NLS, cholic acid was conjugated to the free amino group of the N-terminal cysteine residue of a 13-mer peptide (CGYGPKKKRKVGG; SEQ ID NO: 1) that comprises a nuclear localization signal from SV40 large T-antigen (SEQ ID NO: 2) flanked by linker amino acids and comprising a C-terminal amide. A list of the tested peptides and steroid acid-peptide conjugates for hydrogel formation is found in FIG. 1.

### Dimerization of Steroid Acid-Peptide Conjugates

50 mg of steroid acid-peptide conjugate was resuspended in 200-500 μL of DMSO. 10 eq of N,N-Diisopropylethylamine (DIPEA) was added to the mixture and incubated in a thermomixer at 37° C. with agitation (1000 rpm) overnight, enabling dimerization of the peptides via their terminal cysteine thiol groups. The samples were then photographed to visually document hydrogel formation and subsequently analyzed by UPLC-MS to evaluate dimer formation efficiency. Sample were then further analyzed by light microscopy to characterize the hydrogel materials, and cell toxicity was evaluated via a Cell Toxicity assay (PrestoBlue™). CA-melittin hydrogel was observed to be a more semi-liquid-solid hydrogel (very viscous solution).

### Hydrogel Formation

Specific hydrogel formation was conducted by resuspending the powder of each steroid acid-peptide monomer or dimer conjugate in phosphate buffered saline (PBS) 1X (pH 7.4), pure water, complete cell medium (DMEM), DMSO, or alcohol:water mixtuLes (e.g., methanol:water (40:60)) at room temperature or 37° C. at a concentration between 6-30 mg/mL.

### Cell Cytotoxicity Assay

5000 JIMT1 cells (breast cancer cell line) per well (96-well plate) were plated. After one day, cells were treated with the different steroid acid-peptide conjugates or hydrogels for three days. Cell survival was determined by PrestoBlue™ following the manufacturer's protocol.

### Example 2: Steroid Acid-Peptide Monomer Conjugates Spontaneously Form a Hydrogel

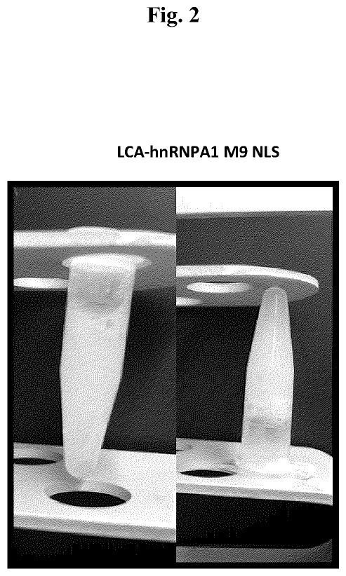

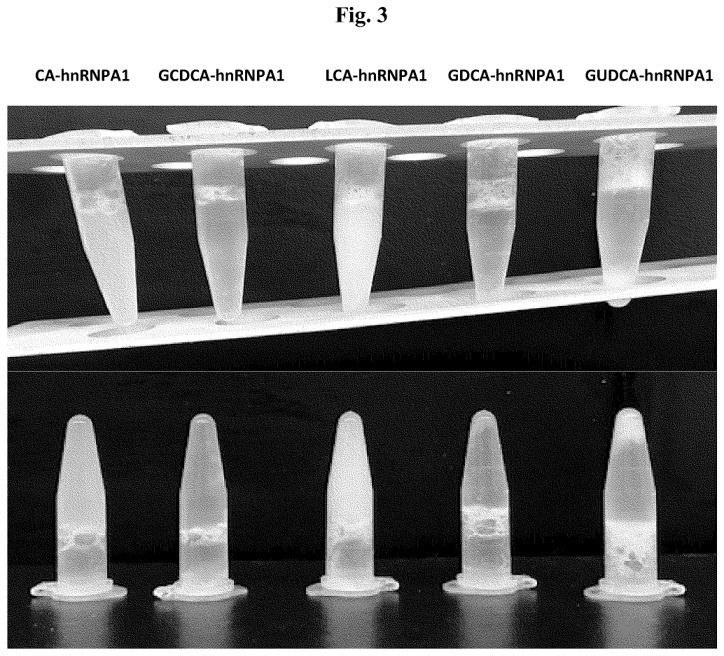

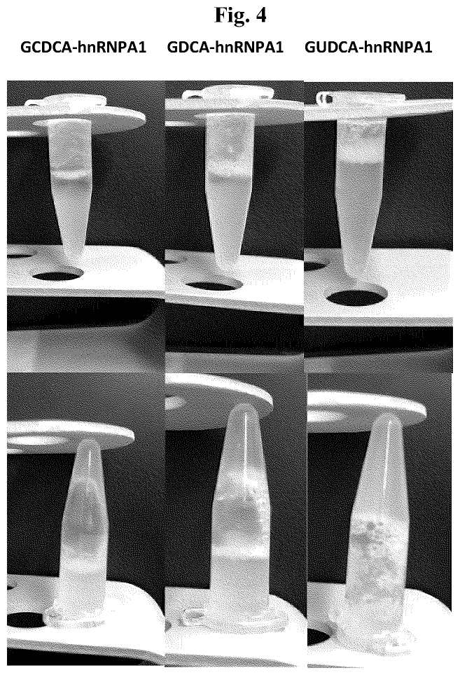

Hydrogel formation was evaluated upon dissolution of the different peptide or steroid acid-peptide conjugates described in FIG. 1 in different solvents. The kinetics of hydrogel formation was found to be dependent on the steroid acid-peptide conjugate, concentration of the steroid acid-peptide conjugate, as well as the solvent used. Hydrogel structure (soft vs. hard) and color/clarity (translucent vs. opaque) varied among the different conditions. Surprisingly, immediately following resuspension of LCA-hnRNPA1 NLS monomers (i.e., hnRNPA1 M9 NLS; SEQ ID NO: 4) (25 mg/mL) in PBS 1X (pH 7.4) at room temperature, a hydrogel spontaneously formed within a matter of seconds, as shown by the inverted tube (FIG. 2). At 20 mg/mL, hnRNPA1 NLS monomers conjugated to either CA, GCDCA, LCA, GDCA, or GUDCA formed a hydrogel after at least 3 hours of incubation in PBS at room temperature (FIG. 3). At 10 mg/mL, all steroid acid-hnRNPA1 NLS monomer conjugates tested formed a hydrogel after 1-2 days in PBS at room temperature (only shown for GCDCA-, GDCA-, GUDCA-hnRNPA1 NLS; FIG. 4). At concentrations lower than 10 mg/mL, all steroid acid-hnRNPA1 NLS conjugates tested formed liquid droplets in PBS at room temperature. Monomers of GWG-SV40NLS conjugated to steroid acids were also shown to form hydrogels (data not shown).

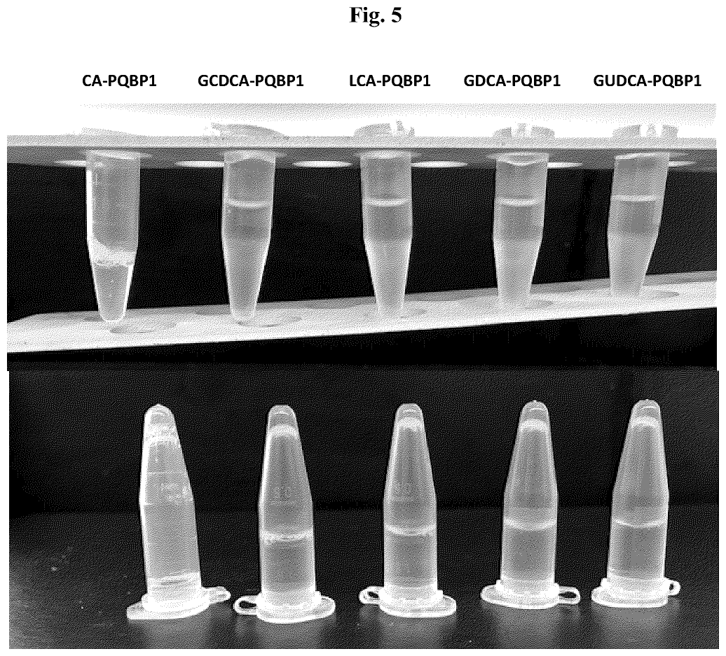

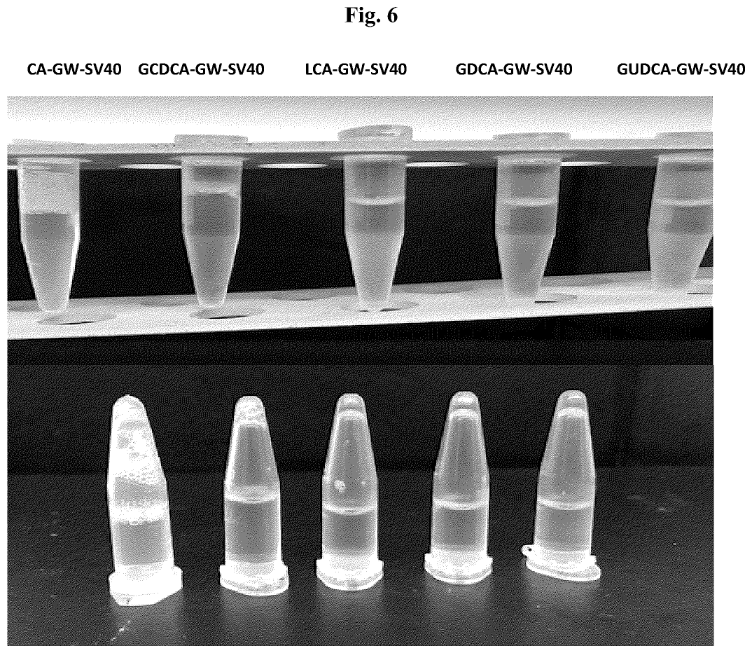

Other steroid acid-peptide monomer conjugates tested in FIG. 1 were not able to form hydrogels at 10-20 mg/mL in PBS after several days of incubation at room temperature. This is shown for steroid acid conjugates of PQBP-1 and GWG-SV40 in FIGS. 5 and 6, respectively.

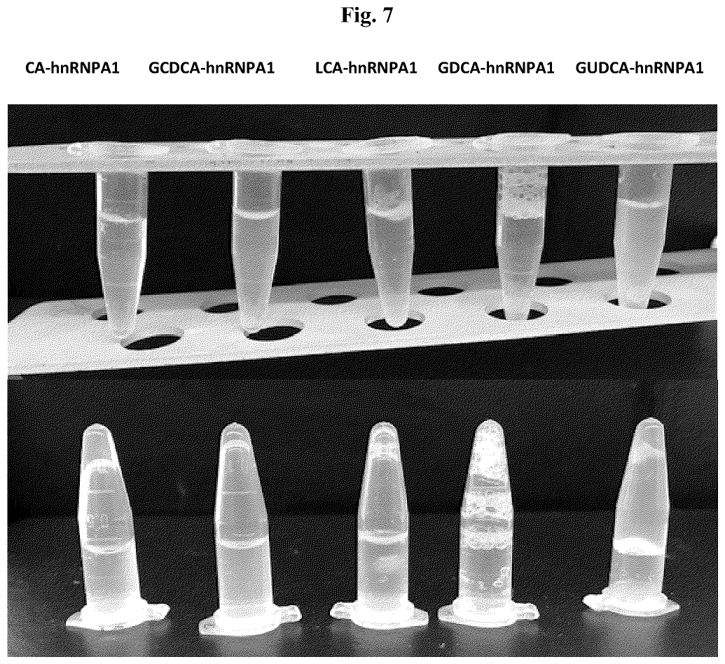

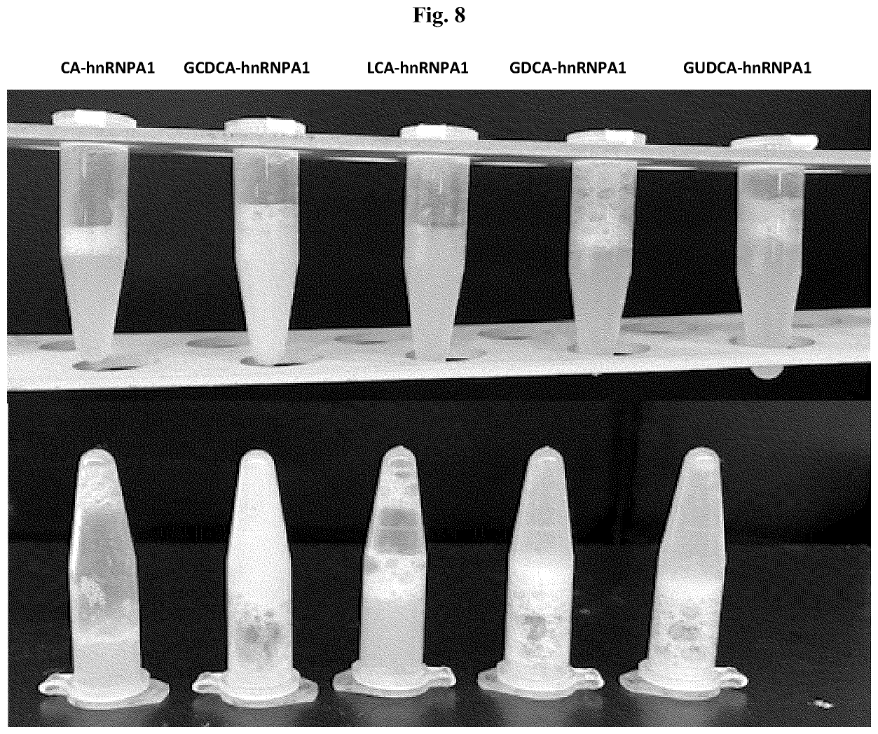

With regards to other solvents tested, a hydrogel was formed when GUDCA-hnRNPA1 NLS monomer was dissolved in pure water at 20 mg/mL after 1 h and up to 3 h at room temperature (FIG. 7). However, after overnight incubation, all steroid acid-hnRNPA1 NLS monomer conjugates tested formed a hydrogel (data not shown). We observed that GCDCA-hnRNPA1 NLS monomer (final concentration 6.67 mg/mL) induced hydrogel formation after 30 min in complete DMEM containing human cells at room temperature and 37° C. Following 2 hours of incubation, GCDCA/GDCA/GUDCA-hnRNPA1 NLS monomer conjugates formed a hydrogel in cell medium at room temperature and 37° C. (FIG. 8). Similar results were seen after overnight incubation.

These data demonstrate the surprising result that different steroid acid-hnRNPA1 NLS monomer conjugates spontaneously form a hydrogel when dissolved in various solvents at different temperatures and concentrations.

### Example 3: Dimerized Steroid Acid-Peptide Conjugates Spontaneously Form a Hydrogel

Hydrogel formation was observed for dimerized steroid acid-peptide conjugates, surprisingly in even steroid acid-peptide conjugates that did not form a hydrogel as monomers. Dimerization was performed as described in Example 1.

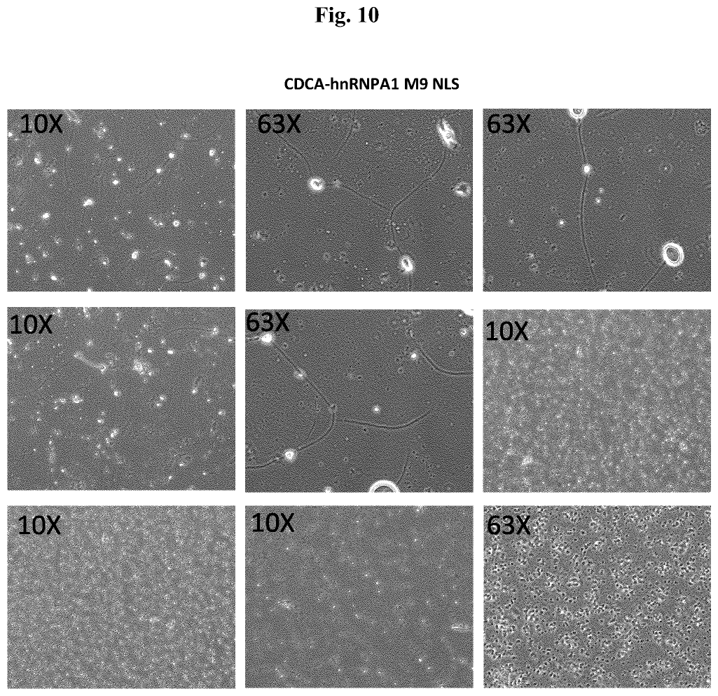

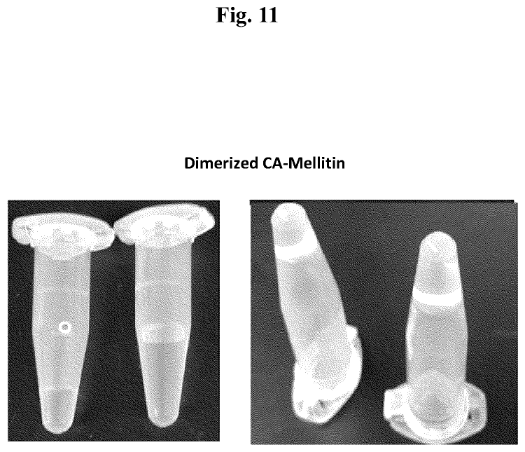

Dimerized CDCA-hnRNPA1 M9 NLS maintained its ability to form a hydrogel. Light microscopy images of the hydrogels formed by CDCA-hnRNPA1 M9 NLS dimers are shown in FIG. 10.

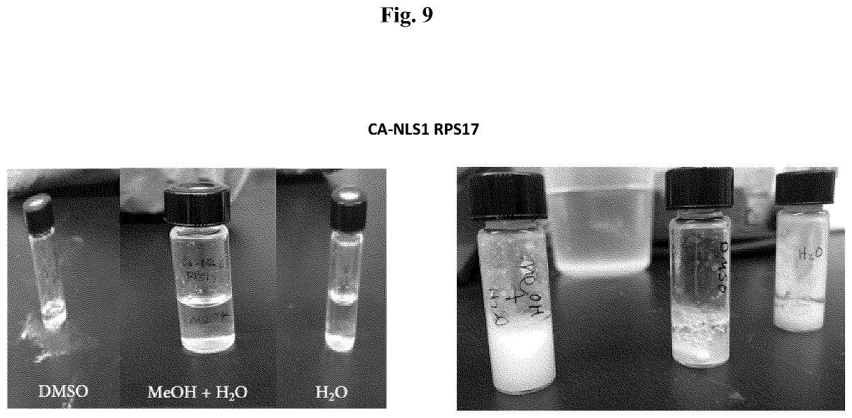

Interestingly, CA-NLS1 RPS17 was not observed to form a hydrogel in its monomer form, yet was able to form a hydrogel in its dimerized form in multiple solvents (FIG. 9).

All steroid acid-peptide conjugates were able to form hydrogels spontaneously following their dimerization reactions (in 100% DMSO). However, addition of water or aqueous solvent consistently enhanced hydrogel formation. Furthermore, dimer-based hydrogels were formed in the presence of different solvents, such as methanol, DMSO, and water.

The peptide melittin is not typically considered as an NLS peptide, but has been shown to exhibit nuclear localization activity (Ogris et al., 2001). Interestingly, monomers of CA-Melittin (SEQ ID NO: 16) were not able to form a hydrogel, but dimerized CA-Melittin was able to form a hydrogel having a softer texture than those formed by other steroid acid-peptide conjugates. Light microscopy images of the hydrogels formed by dimerization of CA-Mellitin are shown in FIG. 12.

### Example 4: Effect of Dimerization of Steroid Acid-Peptide Conjugates on Hydrogel Cytotoxicity

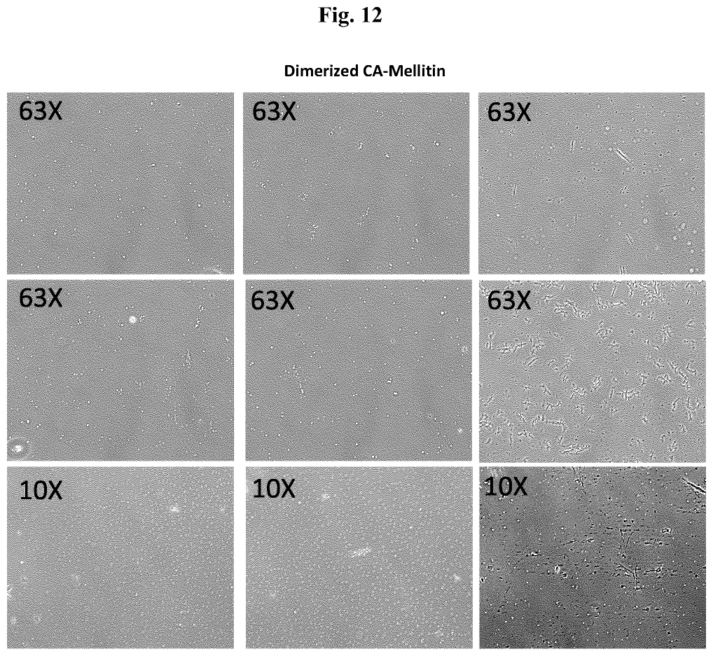

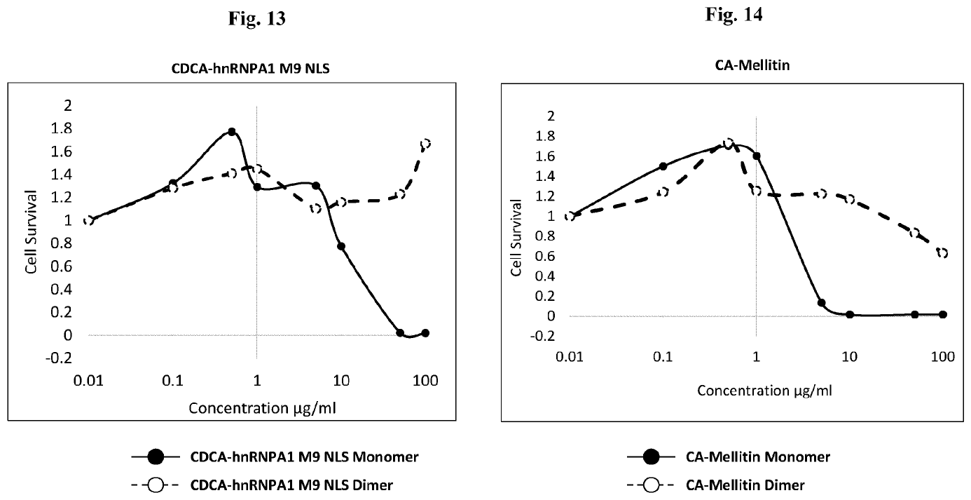

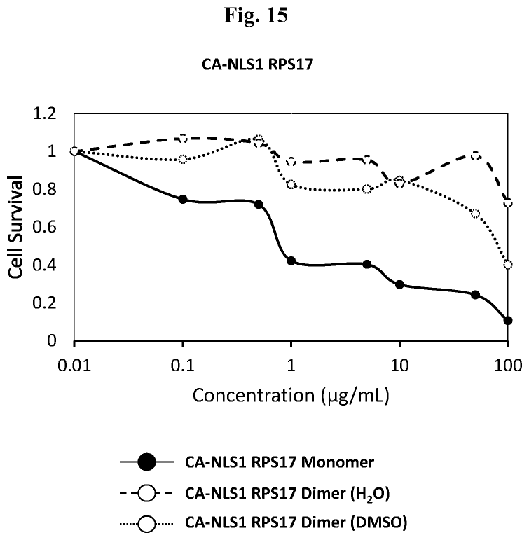

Hydrogels are commonly used for drug delivery and in vivo applications, and therefore assessing their safety profiles is highly pertinent. Cytotoxicity of hydrogels was evaluated via a cytotoxicity assay by incubating cells with hydrogels formed by monomers or steroid acid-peptide conjugates, as described in Example 1. As shown in FIG. 13, hydrogels formed by monomers of CDCA-hnRNPA1 M9 NLS reduced the viability of cells at concentrations higher than 10 μg/mL. Viability was maintained, however, in the presence of hydrogels formed by dimers of CDCA-hnRNPA1 M9 NLS at all concentrations tested up to 100 μg/mL. Similar results were shown with CA-Mellitin, whereby hydrogels formed by dimers of CA-Mellitin were significantly less toxic (FIG. 14). Furthermore, hydrogels formed by dimers of CA-NLS1 RPS17, either in water or DMSO (and heated 100° C. for 2-3 minutes to ensure complete dissolution), were also observed to be significantly less toxic than hydrogels formed by CA-NLS1 RPS17 monomers (FIG. 15).

These data demonstrate the reduced toxicity of hydrogels formed by dimerized steroid acid-peptide conjugates for potential in vivo applications.

### REFERENCES

Beaudoin et al., (2016). ChAcNLS, a novel modification to antibody-conjugates permitting target cell-specific endosomal escape, localization to the nucleus and enhanced total intracellular accumulation. Molecular Pharmaceutics, 13(6): 1915-26.
Ogris et al., (2001). Melittin enables efficient vesicular escape and enhanced nuclear access of nonviral gene delivery vectors. Journal of Biological Chemistry, 276(50): 47550-5.
Sun et al., (2016). Factors influencing the nuclear targeting ability of nuclear localization signals. Journal of Drug Targeting, 24(10): 927-933.

1. A method of forming/producing a hydrogel, the method comprising resuspending/dissolving a peptide covalently conjugated to one or more steroid acid moieties in a solvent until a hydrogel is formed.

2. A method of forming/producing a hydrogel, the method comprising providing a peptide to be modified, covalently conjugating the peptide to one or more steroid acid moieties to produce a steroid acid-peptide conjugate, and resuspending/dissolving the steroid acid-peptide conjugate in a solvent until a hydrogel is formed.

3. The method of claim 1 or 2, wherein the peptide comprises an amino acid sequence comprising at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.

4. The method of any one of claims 1 to 3, wherein the peptide is or comprises a nuclear localisation signal (NLS).

5. The method of any one of claims 1 to 4, wherein the steroid acid is a bile acid.

6. The method of any one of claims 1 to 5, wherein the steroid acid is a primary bile acid or a secondary bile acid.

7. The method of any one of claims 1 to 6, wherein the steroid acid is or comprises cholic acid (CA), chenodeoxycholic acid (CDCA), deoxycholic acid (DCA), lithocholic acid (LCA), glycocholic acid (GCA), taurocholic acid (TCA), glycodeoxycholic acid (CDCA), glycochenodeoxycholic acid (GCDCA), taurodeoxycholic acid (TDCA), glycolithocholic acid (GLCA), taurolithocholic acid (TLCA), taurohyodeoxycholic acid (THDCA), taurochenodeoxycholic acid (TCDCA), ursocholic acid (UCA), tauroursodeoxycholic acid (TUDCA), glycoursodeoxycholic acid (GUDCA), ursodeoxycholic acid (UDCA), or any combination thereof.

8. The method of any one of claims 1 to 7, wherein the steroid acid is or comprises lithocholic acid (LCA), cholic acid (CA), glycochenodeoxycholic acid (GCDCA), glycodeoxycholic acid (GDCA), or glycoursodeoxycholic acid (GUDCA).

9. The method of any one of claims 1 to 8, wherein the steroid acid-peptide conjugate comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 steroid acid moieties.

10. The method of any one of claims 1 to 8 wherein the peptide is conjugated to the steroid acid at a solvent-accessible amine (e.g., primary amine) and/or sulfhydryl groups of the peptide.

11. The method of any one of claims 1 to 10, wherein the peptide is conjugated to the steroid acid via a linker (e.g., bifunctional, trifunctional linker, or multi-functional linker).

12. The method of any one of claims 1 to 11, wherein the peptide is conjugated to the steroid acid-peptide conjugate via an N-terminal or C-terminal cysteine residue.

13. The method of any one of claims 1 to 12, wherein the peptide is or comprises a classical nuclear localisation signal NLS, a non-classical NLS, a hydrophobic PY-NLS, or a basic PY-NLS.

14. The method of any one of claims 1 to 13, wherein the peptide is or comprises a nuclear localisation signal (NLS) which is an NLS from simian vacuolating virus 40 (SV40) large T-antigen, GWG-SV40NLS, NLS2 from ribosomal protein S17 (RPS17), NLS1 from RPS17, NLS3 from RPS17, NLS2 RG RSP17, nucleoplasmin NLS, acidic M9 domain in the heterogeneous nuclear ribonucleoprotein (hnRNP) A1 protein, hnRNPA1 M9 NLS, hnRNP D NLS, hnRNP M NLS, HuR NLS, cMyc NLS, TUS NLS, polyglutamine-binding protein 1 (PQBP1) NLS, the sequence KIPIK in yeast transcription repressor Matα2, PY-NLS, ribosomal NLS, the complex signals of U snRNPs, or a portion of the NLS thereof.

15. The method of any one of claims 1 to 14, wherein the peptide is or comprises a nuclear localisation signal (NLS) comprising an amino acid sequence that is at least 70%, 75%, 80%, 85%, 90%, 95%, 99%, or 100% identical to any one of SEQ ID NOs: 1-15.

16. The method of any one of claims 1 to 15, wherein the peptide is or comprises a nuclear localisation signal (NLS), a portion, or domain of the heterogeneous nuclear ribonucleoprotein (hnRNP) A1 protein.

17. The method of any one of claims 1 to 16, wherein the peptide is or comprises a nuclear localisation signal (NLS) as set forth in SEQ ID NO: 4.

18. The method of any one of claims 1 to 12, wherein the peptide is or comprises Mellitin (SEQ ID NO: 16).

19. The method of any one of claims 1 to 18, wherein the steroid acid-peptide conjugate is a monomer.

20. The method of any one of claims 1 to 19, wherein the steroid acid-peptide conjugate is a dimer or oligomer.

21. The method of any one of claims 1 to 20, wherein the solvent is or comprises an aqueous solvent (e.g., water).

22. The method of any one of claims 1 to 21, wherein the solvent is or comprises a buffered solution (e.g., phosphate buffered saline (PBS) Tris buffered saline (TBS)), DMSO, an alcohol (e.g., ethanol or methanol), or medium.

23. The method of any one of claims 1 to 22, wherein the solvent is at a substantially neutral pH.

24. The method of any one of claims 1 to 23, wherein the solvent is at a pH of 6.5, 6.6, 6.7, 6.8, 6.9, 7.0, 7.1, 7.2, 7.3, 7.4, or 7.5.

25. The method of any one of claims 1 to 24, wherein the hydrogel is produced/formed instantly upon resuspension/dissolution of the steroid acid-peptide conjugate in the solvent.

26. The method of any one of claims 1 to 24, wherein the hydrogel is produced/formed at least 1, 5, 10, 15, 20, 25, 30, 35, 40, 45, 50, 55, or 60 seconds after resuspension/dissolution of the steroid acid-peptide conjugate in the solvent.

27. The method of any one of claims 1 to 24, wherein the hydrogel is produced/formed at least 1, 5, 10, 15, 20, 25, 30, 35, 40, 45, 50, 55, 60, 120, 180, 240, 300, or 360 minutes after resuspension/dissolution of the steroid acid-peptide conjugate in the solvent.

28. The method of any one of claims 1 to 24, wherein the hydrogel is produced/formed at least 6, 8, 10, 12, 14, 16, 18, 20, 22, or 24 hours after resuspension/dissolution of the steroid acid-peptide conjugate in the solvent.

29. The method of any one of claims 1 to 28, wherein the concentration of the steroid acid-peptide conjugate resuspended/dissolved in the solvent or comprised in the hydrogel is at least 0.001, 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL.

30. The method of any one of claims 1 to 29, wherein the hydrogel comprises liquid droplets.

31. The method of any one of claim 1 to 30, further comprising dimerizing the steroid acid-peptide conjugate.

32. A method of forming/producing a hydrogel, the method comprising resuspending/dissolving a peptide in a steroid acid solution, or mixing a steroid acid and peptide in a solvent, until a hydrogel is formed.

33. A hydrogel formed/produced by the method of any one of claims 1 to 32.

34. A hydrogel comprising the steroid acid-peptide conjugate as defined in any one of claims 3 to 20.

35. A steroid acid-peptide conjugate (e.g., in monomeric, dimeric, or oligomeric form) for use in the production/formation of a hydrogel.

36. Use of a steroid acid peptide-conjugate (e.g., in monomeric, dimeric, or oligomeric form) for the production/formation of a hydrogel.

37. The steroid acid-peptide conjugate for use of claim 35 or the use of claim 36, wherein the steroid acid peptide-conjugate is the steroid acid-peptide conjugate defined in any one of claims 3 to 20.

38. A steroid acid-peptide conjugate comprising lithocholic acid (LCA), cholic acid (CA), glycochenodeoxycholic acid (GCDCA), glycodeoxycholic acid (GDCA), or glycoursodeoxycholic acid (GUDCA) conjugated to a nuclear localisation signal (NLS) from heterogeneous nuclear ribonucleoprotein (hnRNP) A1 M9 protein, for use in the production/formation of a hydrogel.

39. The steroid acid-peptide conjugate of claim 38, which is a monomer, a dimer, or an oligomer.

40. A hydrogel produced/formed by conjugating lithocholic acid (LCA), cholic acid (CA), glycochenodeoxycholic acid (GCDCA), glycodeoxycholic acid (GDCA), or glycoursodeoxycholic acid (GUDCA) to a nuclear localisation signal (NLS) from heterogeneous nuclear ribonucleoprotein (hnRNP) A1 M9 protein; and incubating the conjugate to a suitable solvent.

41. A hydrogel comprising a steroid acid-peptide conjugate (e.g., in monomeric, dimeric, or oligomeric form), wherein the steroid acid-peptide conjugate is or comprises lithocholic acid (LCA), cholic acid (CA), glycochenodeoxycholic acid (GCDCA), glycodeoxycholic acid (GDCA), or glycoursodeoxycholic acid (GUDCA) conjugated to a nuclear localisation signal (NLS) from heterogeneous nuclear ribonucleoprotein (hnRNP) A1 M9 protein.


SEQUENCE LISTING (16 sequences)

SEQ ID NO: 1 - protein, 12 aa, synthetic construct
GYGPKKKRKVGG

SEQ ID NO: 2 - protein, 7 aa, synthetic construct
PKKKRKV

SEQ ID NO: 3 - protein, 18 aa, synthetic construct
GWWGYGPKKKRKVGGWWG

SEQ ID NO: 4 - protein, 19 aa, synthetic construct
SNFGPMKGGNFGGRSSGPY

SEQ ID NO: 5 - protein, 19 aa, synthetic construct
SGYGKVSRRGGHQNSYKPY

SEQ ID NO: 6 - protein, 20 aa, synthetic construct
ADREEGKERRHHRREELAPY

SEQ ID NO: 7 - protein, 19 aa, synthetic construct
NKRVCEEIAIIPSKKLRNK

SEQ ID NO: 8 - protein, 19 aa, synthetic construct
SKKLRNKIAGYVTHLMKRI

SEQ ID NO: 9 - protein, 15 aa, synthetic construct
MGRVRTKTVKKAAGG

SEQ ID NO: 10 - protein, 20 aa, synthetic construct
AVKRPAATKKAGQAKKKKLD

SEQ ID NO: 11 - protein, 14 aa, synthetic construct
GYGPAAKRVKLDGG

SEQ ID NO: 12 - protein, 14 aa, synthetic construct
GYGKLKIKRPVKGG

SEQ ID NO: 13 - protein, 19 aa, synthetic construct
NEKRKEKNIKRGGNRFEPY

SEQ ID NO: 14 - protein, 21 aa, synthetic construct
GRFSPMGVDHMSGLSGVNVPG

SEQ ID NO: 15 - protein, 33 aa, synthetic construct
NKRVCEEIAIIPSKKLRNKGSGRIQRGPVRGIS

SEQ ID NO: 16 - protein, 27 aa, synthetic construct
CGIGAVLKVLTTGLPALISWIKRKRQQ